(19) United States
(12) Patent Application Publication
(10) Pub. No.: US 2026/0150174 A1
(43) Pub. Date: May 28, 2026

(54) MULTI-MODE X-RAY EXPOSURE INITIATION DEVICES

(71) Applicants: Gary August Fromberg, Arlington Heights, IL (US); Robert L. Burbury, Springfield, MO (US)

(72) Inventors: Gary August Fromberg, Arlington Heights, IL (US); Robert L. Burbury, Springfield, MO (US)

(21) Appl. No.: 19/384,730

(22) Filed: Nov. 10, 2025

Related U.S. Application Data
(60) Provisional application No. 63/833,412, filed on Nov. 27, 2024.

(51) Int. Cl. H05G 1/38; H05G 1/02

(52) U.S. Cl. CPC H05G 1/38; H05G 1/02

(57) ABSTRACT

Multi-mode x-ray exposure initiation devices are disclosed. An example an apparatus includes: a housing including a front surface, a back surface opposite the front surface, a first side surface, and a second side surface opposite the first side surface, the front surface substantially parallel to the back surface, the first side surface opposite the second side surface; a first button to trigger an X-ray device to prepare for an exposure; a second button to trigger the X-ray device to implement the exposure; and a connector to enable electrical coupling of the first and second buttons to the X-ray device.

### RELATED APPLICATION

This patent claims the benefit of U.S. Provisional Patent Application No. 63/833,412, which was filed on Nov. 27, 2024. U.S. Provisional Patent Application No. 63/833,412 is hereby incorporated herein by reference in its entirety. Priority to U.S. Provisional Patent Application No. 63/833,412 is hereby claimed.

### FIELD OF THE DISCLOSURE

This disclosure relates generally to X-ray machines and, more particularly, to multi-mode X-ray exposure initiation devices.

### BACKGROUND

Preparation/Exposure switches (or Prep/Expose switches for short) are common within X-Ray environments, whether they are used to start an X-ray machine in connection with a veterinary examination, medical procedure, or other application. Prep/Expose switches initiate the chain of events associated with the various support equipment of an X-ray machine (e.g., generators, digital panels, etc.) that result in an image of the subject of interest (e.g., anatomy of an animal or human).

### BRIEF DESCRIPTION OF THE DRAWINGS

FIG. 1 is a perspective view of an example X-ray initiation device constructed in accordance with teachings disclosed herein.

FIG. 2 is a side view of the example floor cradle of the example X-ray initiation device of FIG. 1.

FIG. 3 is a side view of the example Prep/Expose switch of the example X-ray initiation device of FIG. 1.

FIG. 4 is a perspective view of the example Prep/Expose switch of FIG. 1 with the example activation lever removed.

FIG. 5 is an exploded view of the example Prep/Expose switch of FIG. 1.

FIG. 6 is a schematic diagram of the example wiring associated with the example Prep/Expose switch of FIG. 1.

FIG. 7 is an exploded view of the example activation lever and the example pivot rod prior relative to assembly with the example Prep/Expose switch of FIG. 1.

FIG. 8 is a perspective view of an example push-pull connector with an associated coil cord to facilitate connection of the example Prep/Expose switch of FIG. 1 to other components of an X-ray machine.

FIG. 9 is an exploded view of the example floor cradle of FIG. 1.

FIG. 10 illustrates an example foot operation mode of the example Prep/Expose switch of FIG. 1.

FIG. 11 illustrates an example hands-free operation mode of the example Prep/Expose switch of FIG. 1.

FIG. 12 illustrates an example hand operation mode of the example Prep/Expose switch of FIG. 1.

In general, the same reference numbers will be used throughout the drawing(s) and accompanying written description to refer to the same or like parts. The figures are not necessarily to scale.

### DETAILED DESCRIPTION

Prep/Expose switches (also referred to herein as a Preparation/Exposure (Prep/Expose) initiation devices) are commonly used across the X-ray industry. Such devices typically include a two-stage button associated with two sets of normally open contacts. The first set of contacts close to activate the “preparation” stage of an X-ray machine in which an X-ray generator is activated and begins to spin up the X-ray tube rotor to prepare for an X-ray exposure. The second set of contacts subsequently close to cause the generator to initiate an X-ray exposure (e.g., the “exposure” stage) and produce the high voltage needed within the X-ray tube to cause X-rays to be emitted towards the subject of interest that are then captured by an X-ray detector or film. Some known Prep/Expose switches are designed to be handheld. Such hand-operated switches are often cylindrically shaped to be gripped by an operator with a two-stage button at one end that can be pressed by the operator's thumb. Other known Prep/Expose switches are designed to be foot operated. Such foot-operated devices are structured with a flat bottom that is to rest on the examination room floor with an upward facing foot pedal that can be stepped on by an operator.

Unlike known Prep/Expose switches, example Prep/Expose switches disclosed herein can be operated in multiple modes including a hand operated mode, a foot operated mode, and a hands-free (e.g., underarm) mode. The different modes are made possible by the structure of the disclosed switch that can be handheld for hand operation and easily operated under an operator's arm (without the dexterity available using hands and fingers) when the operator needs their hands free (e.g., if the operator needs his or her hands to hold the subject to be X-rayed). Further, in some examples, the switch is packaged with an associated floor cradle that can support the switch on the floor for operation with an operator's foot.

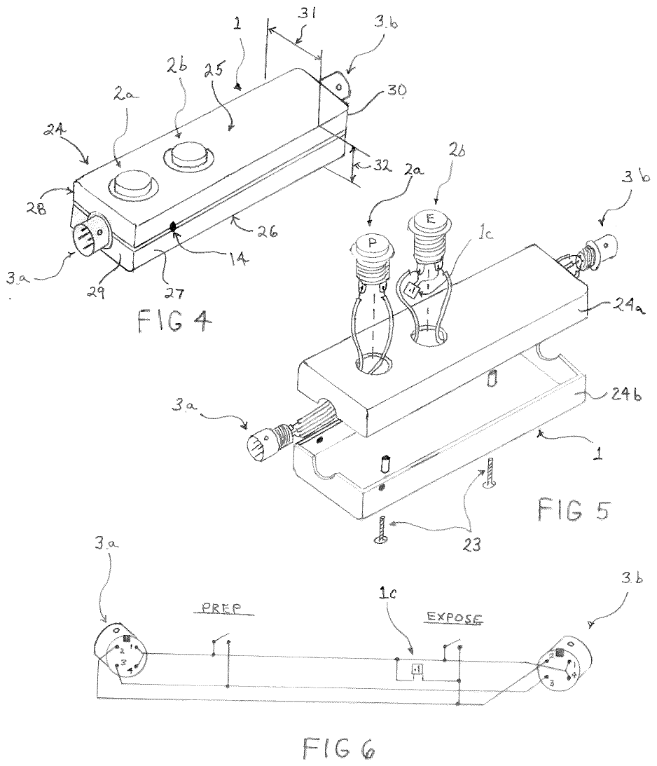

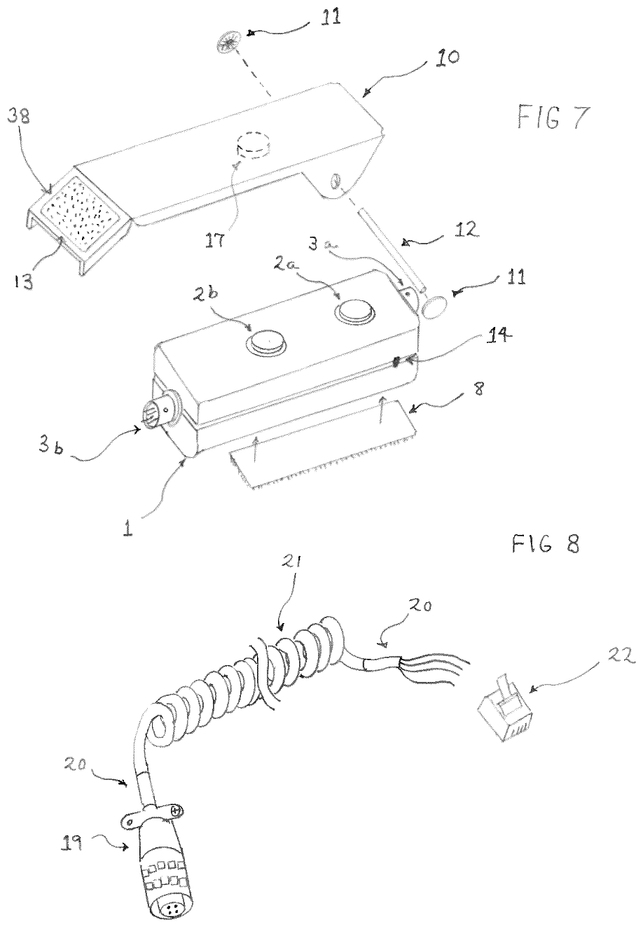

Referring now to the drawings, FIGS. 1, 3, 4, 5, and 7 show different views of an example X-ray initiation device 33 constructed in accordance with teachings disclosed herein. As shown in the illustrated example of FIG. 1, the X-ray initiation device 33 includes an example Prep/Expose switch 1 (e.g., an X-ray exposure switch assembly) that can rest on and/or be supported by an example floor cradle 34. In some examples, the Prep/Expose switch 1 includes a substantially rigid hollow rectangular, elongated plastic housing 24 (e.g., enclosure). In some examples, as shown most clearly in the illustrated example of FIG. 5, the housing 24 include two halves 24a, 24b for ease of assembly and/or disassembly. In this example, the two halves 24a, 24b of the housing 24 are secured together by two screws 23. In other examples, any other suitable number of screws 23 may be employed. In other examples, the two halves 24a, 24b snap together without the use of screws.

In some examples, the housing 24 includes a top surface 25 (e.g., front surface) that is substantially parallel to a bottom surface 26 (e.g., back surface). Further, in this example, the housing 24 includes a first side surface 27 that is substantially parallel to a second side surface 28. In some examples, the top and bottom surfaces 25, 26 are substantially perpendicular to the first and second side surfaces 27, 28. As used herein, substantially parallel means within 10 degrees of exactly parallel. Further, as used herein, substantially perpendicular means within 10 degrees of exactly perpendicular. As shown in the illustrated example, each of the four surfaces 25, 26, 27, 28 of the housing 24 extend a length of the housing 24 between opposing first and second ends 29, 30.

In some examples, the top and bottom surfaces 25, 26 have a first width 31 and the two side surfaces 27, 28 have a second width 32 that is different from the first width. More particularly, in this example, the first width 31 is greater than the second width 32. In other examples, the first width 31 is less than the second width 32. In other examples, the first width 31 is approximately equal to the second width 32. Regardless of the relationship between the first and second widths 31, 32, in some examples, both widths 31, 32 are less than the length of the enclosure. In some examples, the length is multiple times (e.g., at least twice) the size of at least one of the first width 31 or the second width 32. In some examples, the first and second widths are less than or equal to 2 inches (e.g., less than or equal to 1.5 inches, less than or equal to 1.0 inch) and the length is less than or equal to 6 inches (e.g., less than or equal to 5.5 inches, less than or equal to 5.0 inches).

As shown in the illustrated examples, the housing 24 contains, supports, and/or carries two normally open tactile pushbuttons 2a, 2b (switches) as shown in FIGS. 3, 4, 5 and 7. In some examples, the pushbuttons 2a, 2b are identical. In this example, the first pushbutton 2a is closer to the first end 29 than the second pushbutton 2b is to the first end. Further, the first pushbutton 2a is to operate as the “prep” button (hence the label of “P” in FIG. 5) and the second pushbutton 2b is to operate as the “expose” button (hence the label of “E” in FIG. 5. That is, as described further below, in operation the first pushbutton 2a is pressed first and the second pushbutton 2b is pressed second.

In some examples, the housing 24 contains, supports, and/or carries first and second connectors 3a, 3b. In this example, the connectors 3a, 3b are 4-pin male, round, tubular connectors. As shown, the first and second connectors 3a, 3b are located at the respective first and second ends 29, 30 of the housing 24. These two connectors 3a, 3b are wired in parallel as shown in FIG. 6 in conjunction with the two pushbuttons 2a, 2b and will serve as exit points (e.g., connection ports) for those normally open contacts. The two connectors 3a, 3b and their locations in the illustrated example serve as convenient access points for the operator depending on the particular mode in which the Prep/Expose switch 1 is to be used (e.g., either as a foot operated device or as a hand operated device). This gives the operator a choice for the most convenient connector 3a, 3b to use for the application and the X-ray image to be taken.

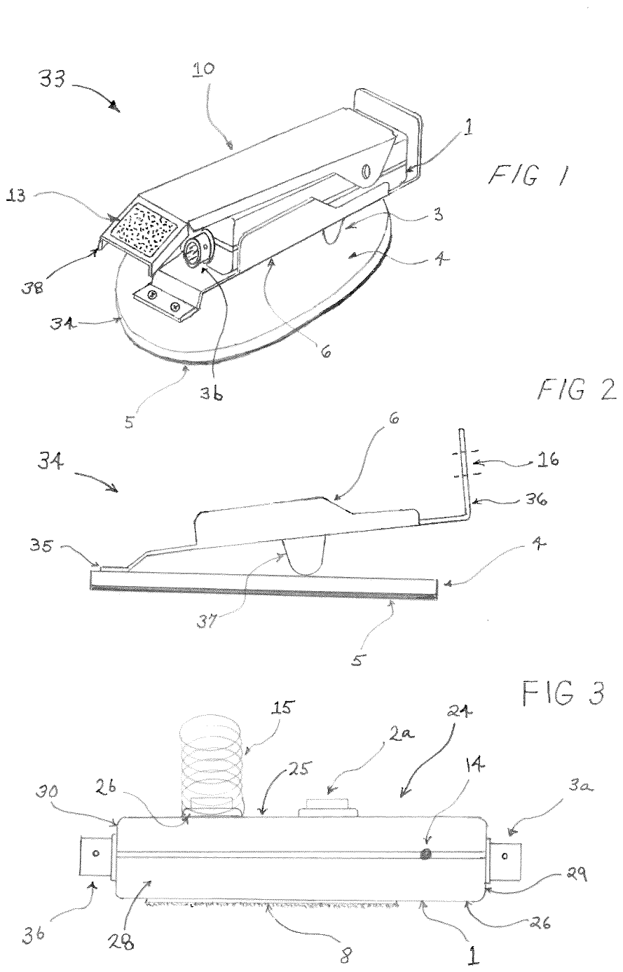

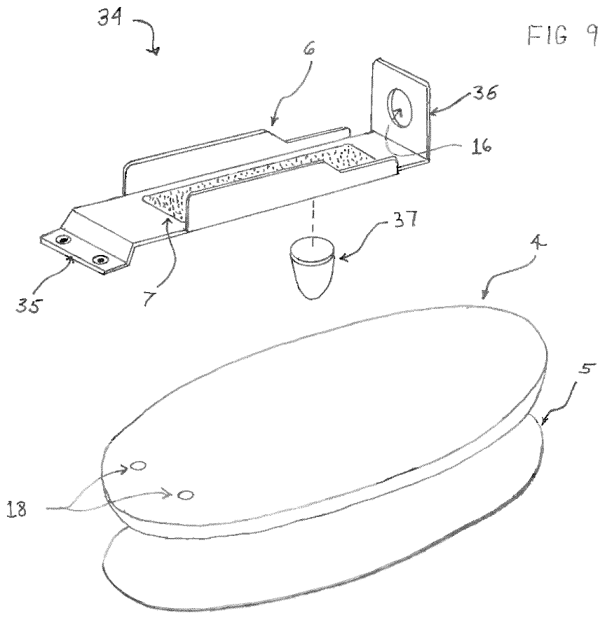

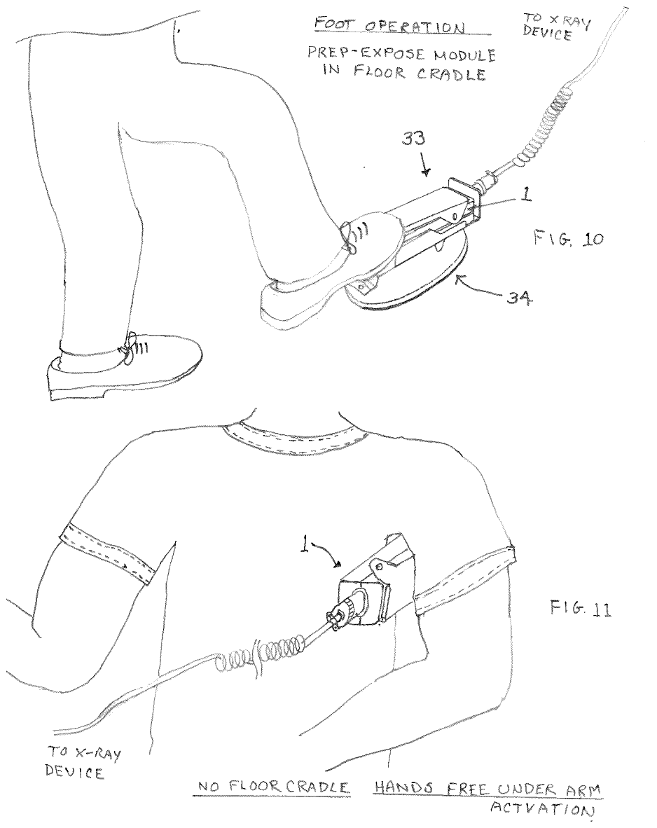

FIGS. 2 and 9 illustrate the example floor cradle 34 that may be employed to hold the Prep/Expose switch 1 for use in the foot-operated mode. FIG. 2 shows a side view of the floor cradle 34 and FIG. 9 shows an exploded view. As shown in the illustrated example, the floor cradle 34 includes a support bracket 6 attached to a base plate 4. In some examples, both the base plate 4 and the support bracket 6 are made of metal. In other examples, at least one of the base plate 4 or the support bracket 6 are made of a different material (e.g., a plastic, a ceramic, etc.). In some examples, the base plate 4 is a round plate that is approximately 6 inches in diameter and approximately 0.25 inches thick. However, in other examples, the base plate 4 can have a different shape (e.g., rectangular, oval, etc.), have a different size (e.g., larger and/or smaller than 6 inches), and/or a different thickness (e.g., larger or smaller than 0.25 inches). In this example, the base plate 4 has a non-skid neoprene rubber pad 5 glued to the bottom so that when used as a floor operated device the Prep/Expose switch 1 in combination with the floor cradle is less likely to slip.

In this example, the support bracket 6 is mounted to the base plate 4 at a first end 35 by way of two threaded fasteners (e.g., machine screws) that extend through corresponding threaded holes 18. In other examples, any other suitable number of fasteners may be used. In other examples, the support bracket 6 is attached to the base plate 4 using any other suitable technique (e.g., an adhesive, welding, etc.). In some examples, the support bracket 6 is an integral extension of the base plate 4.

As shown in the illustrated example, the support bracket 6 is supported at a slanted angle relative to the base plate 4 such that a second end 36 of the bracket 6 is spaced apart from the base plate 4. In some examples, the angle between the bracket 6 and the base plate 4 is maintained by means of a bumper 37. In this example, the bumper 37 is attached to the bracket 6. In other examples, the bumper 37 is attached to the base plate 4. In some examples, the bumper 37 is made of rubber. However, in other examples any other suitable material may be used. In some examples, the Prep/Expose switch 1 is to rest on (e.g., be supported by) the bracket 6. As such, the angle of the support bracket 6 relative to the base plate 4 serves to hold the Prep/Expose switch 1 at an angle relative to the floor on which the floor cradle 34 is positioned.

As shown in the illustrated example, the support bracket 6 includes a large hole 16 in a flange at the second end 36 that serves as an exit point for the second connector 3a when the Prep/Expose switch 1 is used in the floor operation mode. That is, in some examples, the second connector 3a is accessible through the hole 16 when the bracket is holding (e.g., supporting) the switch 1. In some examples, the upward facing surface of the bracket 6 includes a first side 7 of a hook and loop fastener (e.g., a VELCRO™ pad) that selectively attaches to a second side 8 of the hook and loop fastener on the bottom surface 26 of the housing 24 of the Prep/Expose switch 1. In this manner, the switch 1 can be held in place when inserted into the bracket 6 while still enabling the relatively easy removal of the switch 1 by an operator without the need for any tools.

In some examples, an activation lever 10 defined by a plate that is pivotably coupled to the housing 24 of the Prep/Expose switch 1 via a pin or rod 12 extending through holes 14 in the housing 24 adjacent the second end 30. In some examples, the activation lever is made of metal. However, any other suitable material may alternatively be used. In some examples, the rod 12 is held in place by press on captive nuts 11 at each end of the rod. In this example, the rod 12 is approximately 0.125 inches in diameter. However, in other examples, the rod can be any other suitable size.

Rotation of the activation lever 10 about the rod 12 enables the lever 10 to move relative to the top surface 25 of the housing 24 where the pushbuttons 2a, 2b are located. Inasmuch as the activation lever 10 rotates about an axis proximate the second end 30 of the housing 24 and the first pushbutton 2a is closer to the second end 30, the activation lever 10 is closer to the first pushbutton 2a than the second pushbutton 2b when the lever 10 is angled relative to the housing 24. As a result, when the lever 10 is pushed towards the housing 24 (as it is rotated about the rod 12) the lever 10 will come into contact with the first pushbutton 2a before coming into contact with the second pushbutton 2b. Furthermore, in some examples, a protrusion 17 (e.g., a round rubber pad) is attached (e.g., glued) under the activation lever 10 (e.g., on the underside of the lever plate that faces toward the housing 24) to engage with the first pushbutton 2a that corresponding to the “Prep” button. This ensures that the “Prep” button is pressed before the “Expose” button (e.g., the second pushbutton 2b) is pressed. That is, in some examples, the lever 10 can rotate relative to the housing 24 between (1) a first position in which both the first and second pushbuttons 2a, 2b are unpressed, (2) a second position in which the first pushbutton 2a is pressed while the second pushbutton 2b is unpressed, and (3) a third position in which both the pushbuttons 2a, 2b are pressed. In some examples, a clip or ring (e.g., a safe mode holder) can be positioned adjacent (e.g., wrap around at least part of) the second end 29 of the housing so as to be between the top surface 25 of the housing 24 and the activate lever 10. In some such examples, the clip or ring is thick enough to function as a physical stop that blocks or prevents the activation lever 10 from being pressed down sufficiently to press the pushbuttons 2a, 2b, thereby preventing the Prep/Expose switch 1 from being inadvertently pressed to unintentionally activate an X-ray exposure.

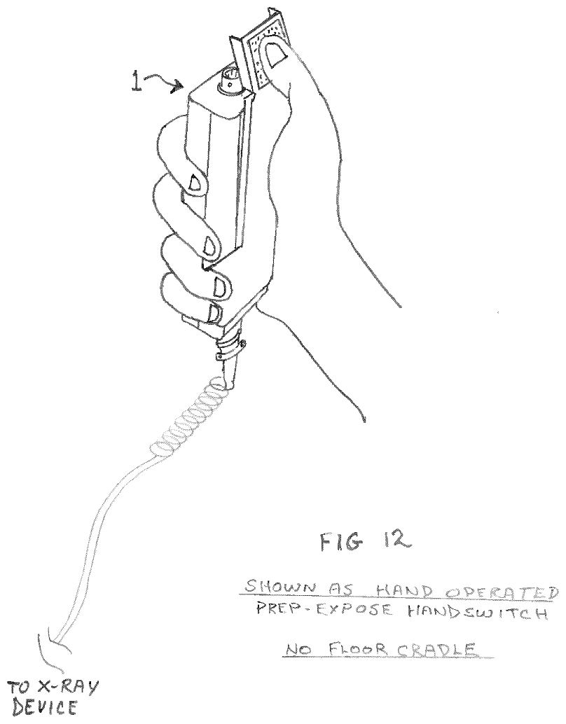

In some examples, a return spring 15 is provided to urge the activation lever 10 away from the housing 24 so that the pushbuttons 2a, 2b are maintained in a non-active, normally open state unless a force is applied to the lever 10 that overcomes the force of the spring 15. That is, the spring 15 prevents the pushbuttons 2a, 2b from being pressed unless, for example, an operator pushes down on the lever with their foot as shown in FIG. 10, forces the lever 10 towards the pushbuttons 2a, 2b under their arm as shown in FIG. 11, or forces the lever 10 towards the pushbuttons 2a, 2b with their hand as shown in FIG. 12. In some examples, the spring 15 is a coil spring that extends between the top surface 25 of the housing 24 and the underside of the main plate of the activation lever 10. In some such examples, the spring 15 wraps around the second pushbutton 2b as shown in FIG. 3. In other examples, the spring 15 is positioned around the first pushbutton 2a. In some such examples, the spring 15 also extends around the protrusion 17 that is positioned to be in alignment with the first pushbutton 2a.

In some examples, the activation lever 10 includes a flange 38 at its free end (e.g., the end distal to the rod 12) that is angled relative to the main plate of the lever 10. In some examples, the flange 38 extends beyond the second end 30 of the housing 24. In some examples, the angle of the flange 38 of the lever 10 relative to the housing 24 in combination with the angle of the support bracket 6 of the cradle 34 relative to the base plate 4 positions the flange 38 at an angle relative to the floor that can easily be pressed down by the foot of an operator as shown in FIG. 10. In some examples, the flange 38 has a non-slip pad 13 disposed thereon to provide greater friction or grip when an operator attempts to push down on the lever 10 with their foot.

FIG. 6 is a schematic diagram of example wiring associated with the example Prep/Expose switch of FIG. 1. In this example, the wiring includes a 0.1 microfarad (uf) capacitor 1c across the “Expose” switch (associated with the second pushbutton 2b) for contact bounce. In other examples, a different capacitor may be used. In some examples, the capacitor 1c is omitted.

FIG. 8 is a perspective view of an example push-pull connector 19 with an associated coil cord 21 to facilitate connection of the example Prep/Expose switch 1 of FIG. 1 to other components of an X-ray machine. In some examples, the coil cord 21 is a four-conductor 26ga coil cord cable that can be any suitable length. In some examples, the Prep/Expose switch 1 and the floor cradle 34 are provided in a kit along with multiple coil cords 21 of different lengths. For instance, in some examples, both a 1-foot cable and a 3-foot cable are provided in the kit. In some such examples, the shorter (e.g., 1-foot) cable is suitable when used in conjunction with small portable handheld X-ray generators that can be used by an operator as a hand switch and/or in a hands-free mode by placing the Prep/Expose switch 1 (and associated lever 10) under their arm so that both hands are free to manipulate the portable X-ray machine and/or the subject to be imaged by the X-ray machine. In some examples, the longer (e.g., 3-foot) cable is suitable when used by an operator in the foot operated mode in which the Prep/Expose switch 1 is placed on the floor within the floor cradle 34.

In some examples, whether one or more cables are provided, each cable is supplied with a push-pull connector 19 (e.g., a female connector) that is capable of electrically coupling with either of the connectors 3a, 3b at either end 29, 30 of the Prep/Expose switch 1. Further, in some examples, a small piece of glued heat shrink tubing 20 is provided to secure the push-pull connectors 19 to the corresponding coil cords 21. In some examples, the other end of the coil cord 21 will have a separate connector 22 installed to electrically couple with X-ray equipment (e.g., an X-ray machine). Different manufacturers of X-ray equipment use different types of connectors. Thus, the separate connector 22 shown in FIG. 8 is for purposes of illustration only and any other suitable type of connector may alternatively be employed.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc., may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, or (7) A with B and with C. As used herein in the context of describing structures, components, items, objects and/or things, the phrase “at least one of A and B” is intended to refer to implementations including any of (1) at least one A, (2) at least one B, or (3) at least one A and at least one B. Similarly, as used herein in the context of describing structures, components, items, objects and/or things, the phrase “at least one of A or B” is intended to refer to implementations including any of (1) at least one A, (2) at least one B, or (3) at least one A and at least one B. As used herein in the context of describing the performance or execution of processes, instructions, actions, activities, etc., the phrase “at least one of A and B” is intended to refer to implementations including any of (1) at least one A, (2) at least one B, or (3) at least one A and at least one B. Similarly, as used herein in the context of describing the performance or execution of processes, instructions, actions, activities, etc., the phrase “at least one of A or B” is intended to refer to implementations including any of (1) at least one A, (2) at least one B, or (3) at least one A and at least one B.

As used herein, singular references (e.g., “a”, “an”, “first”, “second”, etc.) do not exclude a plurality. The term “a” or “an” object, as used herein, refers to one or more of that object. The terms “a” (or “an”), “one or more”, and “at least one” are used interchangeably herein. Furthermore, although individually listed, a plurality of means, elements, or actions may be implemented by, e.g., the same entity or object. Additionally, although individual features may be included in different examples or claims, these may possibly be combined, and the inclusion in different examples or claims does not imply that a combination of features is not feasible and/or advantageous.

As used herein, unless otherwise stated, the term “above” describes the relationship of two parts relative to Earth. A first part is above a second part, if the second part has at least one part between Earth and the first part. Likewise, as used herein, a first part is “below” a second part when the first part is closer to the Earth than the second part. As noted above, a first part can be above or below a second part with one or more of: other parts therebetween, without other parts therebetween, with the first and second parts touching, or without the first and second parts being in direct contact with one another.

As used in this patent, stating that any part (e.g., a layer, film, area, region, or plate) is in any way on (e.g., positioned on, located on, disposed on, or formed on, etc.) another part, indicates that the referenced part is either in contact with the other part, or that the referenced part is above the other part with one or more intermediate part(s) located therebetween.

As used herein, connection references (e.g., attached, coupled, connected, and joined) may include intermediate members between the elements referenced by the connection reference and/or relative movement between those elements unless otherwise indicated. As such, connection references do not necessarily infer that two elements are directly connected and/or in fixed relation to each other. As used herein, stating that any part is in “contact” with another part is defined to mean that there is no intermediate part between the two parts.

Unless specifically stated otherwise, descriptors such as “first,” “second,” “third,” etc., are used herein without imputing or otherwise indicating any meaning of priority, physical order, arrangement in a list, and/or ordering in any way, but are merely used as labels and/or arbitrary names to distinguish elements for ease of understanding the disclosed examples. In some examples, the descriptor “first” may be used to refer to an element in the detailed description, while the same element may be referred to in a claim with a different descriptor such as “second” or “third.” In such instances, it should be understood that such descriptors are used merely for identifying those elements distinctly within the context of the discussion (e.g., within a claim) in which the elements might, for example, otherwise share a same name.

As used herein, “approximately” and “about” modify their subjects/values to recognize the potential presence of variations that occur in real world applications. For example, “approximately” and “about” may modify dimensions that may not be exact due to manufacturing tolerances and/or other real world imperfections as will be understood by persons of ordinary skill in the art. For example, “approximately” and “about” may indicate such dimensions may be within a tolerance range of +/−10% unless otherwise specified herein.

Further examples and combinations thereof include the following:

Example 1 includes an apparatus comprising a housing including a front surface, a back surface opposite the front surface, a first side surface, and a second side surface opposite the first side surface, the front surface substantially parallel to the back surface, the first side surface opposite the second side surface, a first button to trigger an X-ray device to prepare for an exposure, a second button to trigger the X-ray device to implement the exposure, and a connector to enable electrical coupling of the first and second buttons to the X-ray device.

Example 2 includes the apparatus of example 1, wherein the front surface, the back surface, the first side surface, and the second side surface extend a length of the housing between a first end and a second end, the front and back surfaces have a first width that is less than the length, the first and second side surfaces have a second width that is less than the length.

Example 3 includes the apparatus of example 2, wherein the first width is greater than the second width, and the length is more than twice the first width.

Example 4 includes the apparatus of any one or more of examples 2-3, wherein the first and second widths are less than 2 inches, and the length is less than 6 inches.

Example 5 includes the apparatus of any one or more of examples 2-4, wherein the connector is accessible at one of the first end or the second end.

Example 6 includes the apparatus of example 5, wherein the connector is a first connector accessible at the first end of the housing, the apparatus including a second connector accessible at the second end of the housing, either the first connector or the second connector to enable electrical coupling of the first and second buttons to the X-ray device.

Example 7 includes the apparatus of any one or more of examples 1-6, including an activation lever pivotably coupled to the housing, the activation lever to rotate relative to the front surface of the housing to any one of a first position, a second position, or a third position, both the first and second buttons to be unpressed when the activation lever is in the first position, the first button to pressed and the second button to be unpressed when the activation lever is in the second position, both the first and second buttons to be pressed when the activation lever is in the third position, the activation lever closer to the front surface when in the third position than when in the second position, the activation lever closer to the front surface when in the second position than when in the first position.

Example 8 includes the apparatus of example 7, wherein the activation lever includes a plate that is pivotably coupled to the housing via a rod extending between the first and second side surfaces, the rod substantially parallel to the front surface and substantially parallel to the plate, the first and second buttons on the front surface such that the plate moves towards the first and second buttons as the activation lever is moved from the first position to the first position.

Example 9 includes the apparatus of example 8, wherein the rod is proximate an end of the housing, the first button closer to the end of the housing than the second button is to end of the housing.

Example 10 includes the apparatus of example 9, wherein the end is a first end of the housing, the apparatus including a flange extending from the plate, the flange angled relative to the plate, the flange extending beyond a second end of the housing.

Example 11 includes the apparatus of any one or more of examples 8-10, including a protrusion on an underside of the plate facing towards the front surface of the housing, the protrusion to engage with the first button when the activation lever is moved towards the third position.

Example 12 includes the apparatus of any one or more of examples 7-11, including a spring to urge the activation lever toward the first position.

Example 13 includes the apparatus of example 12, wherein the spring is a coil spring that wraps around the second button and extends between the front surface of the housing and the activation lever.

Example 14 includes the apparatus of any one or more of examples 7-13, including a base plate to rest on a floor, and a bracket attached to the base plate, the housing selectively removable from or insertable into the bracket, the back surface of the housing to face towards and be supported by an upward facing surface of the bracket when the housing is inserted into the bracket.

Example 15 includes the apparatus of example 14, wherein the bracket is to hold the housing at an angle relative to the floor.

Example 16 includes the apparatus of any one or more of examples 14-15, including a hook and loop fastener to secure the back surface of the housing adjacent the upward facing surface of the bracket.

Example 17 includes the apparatus of any one or more of examples 14-16, including a bumper on a downward facing surface of the bracket, the bumper to hold the bracket at an angle relative to the base plate.

Example 18 includes the apparatus of any one or more of examples 14-17, wherein the bracket includes a first end attached to the base plate and a second end defined by a flange, the flange to extend away from the base plate, the connector at an end of the housing, the end of the housing to face towards the flange, the connector accessible through a hole in the flange.

Example 19 includes an apparatus comprising an elongate housing having a length extending between a first end and a second end, a first button to trigger X-ray equipment to prepare for an exposure, a second button to trigger the X-ray equipment to implement the exposure, the first and second buttons spaced apart along the length of the elongate housing, a first connector at the first end of the housing, and a second connector at the second end of the housing, either of the first or second connectors to electrical couple with a third connector on a cable that is to be electrically coupled to the X-ray equipment.

Example 20 includes an apparatus comprising an X-ray exposure switch assembly including a switch to trigger operation of an X-ray machine, a connector electrical coupled to the switch, the connector to enable a cable to connect the X-ray exposure switch assembly to the X-ray machine, and a housing to carry the switch and the connector, and a cradle to support the X-ray exposure switch assembly on a floor to facilitate an operator to press the switch with a foot, the X-ray exposure switch assembly manually removable from the cradle by the operator without use of tools, the switch capable of being pressed by the operator when the X-ray exposure switch assembly is separated from the cradle.

The following claims are hereby incorporated into this Detailed Description by this reference. Although certain example systems, apparatus, articles of manufacture, and methods have been disclosed herein, the scope of coverage of this patent is not limited thereto. On the contrary, this patent covers all systems, apparatus, articles of manufacture, and methods fairly falling within the scope of the claims of this patent.

What is claimed is:

1. An apparatus comprising:
a housing including a front surface, a back surface opposite the front surface, a first side surface, and a second side surface opposite the first side surface, the front surface substantially parallel to the back surface, the first side surface opposite the second side surface;
a first button to trigger an X-ray device to prepare for an exposure;
a second button to trigger the X-ray device to implement the exposure; and
a connector to enable electrical coupling of the first and second buttons to the X-ray device.

2. The apparatus of claim 1, wherein the front surface, the back surface, the first side surface, and the second side surface extend a length of the housing between a first end and a second end, the front and back surfaces have a first width that is less than the length, the first and second side surfaces have a second width that is less than the length.

3. The apparatus of claim 2, wherein the first width is greater than the second width, and the length is more than twice the first width.

4. The apparatus of claim 2, wherein the first and second widths are less than 2 inches, and the length is less than 6 inches.

5. The apparatus of claim 2, wherein the connector is accessible at one of the first end or the second end.

6. The apparatus of claim 5, wherein the connector is a first connector accessible at the first end of the housing, the apparatus including a second connector accessible at the second end of the housing, either the first connector or the second connector to enable electrical coupling of the first and second buttons to the X-ray device.

7. The apparatus of claim 1, including an activation lever pivotably coupled to the housing, the activation lever to rotate relative to the front surface of the housing to any one of a first position, a second position, or a third position, both the first and second buttons to be unpressed when the activation lever is in the first position, the first button to pressed and the second button to be unpressed when the activation lever is in the second position, both the first and second buttons to be pressed when the activation lever is in the third position, the activation lever closer to the front surface when in the third position than when in the second position, the activation lever closer to the front surface when in the second position than when in the first position.

8. The apparatus of claim 7, wherein the activation lever includes a plate that is pivotably coupled to the housing via a rod extending between the first and second side surfaces, the rod substantially parallel to the front surface and substantially parallel to the plate, the first and second buttons on the front surface such that the plate moves towards the first and second buttons as the activation lever is moved from the first position to the first position.

9. The apparatus of claim 8, wherein the rod is proximate an end of the housing, the first button closer to the end of the housing than the second button is to end of the housing.

10. The apparatus of claim 9, wherein the end is a first end of the housing, the apparatus including a flange extending from the plate, the flange angled relative to the plate, the flange extending beyond a second end of the housing.

11. The apparatus of claim 8, including a protrusion on an underside of the plate facing towards the front surface of the housing, the protrusion to engage with the first button when the activation lever is moved towards the third position.

12. The apparatus of claim 7, including a spring to urge the activation lever toward the first position.

13. The apparatus of claim 12, wherein the spring is a coil spring that wraps around the second button and extends between the front surface of the housing and the activation lever.

14. The apparatus of claim 7, including:
a base plate to rest on a floor; and
a bracket attached to the base plate, the housing selectively removable from or insertable into the bracket, the back surface of the housing to face towards and be supported by an upward facing surface of the bracket when the housing is inserted into the bracket.

15. The apparatus of claim 14, wherein the bracket is to hold the housing at an angle relative to the floor.

16. The apparatus of claim 14, including a hook and loop fastener to secure the back surface of the housing adjacent the upward facing surface of the bracket.

17. The apparatus of claim 14, including a bumper on a downward facing surface of the bracket, the bumper to hold the bracket at an angle relative to the base plate.

18. The apparatus of claim 14, wherein the bracket includes a first end attached to the base plate and a second end defined by a flange, the flange to extend away from the base plate, the connector at an end of the housing, the end of the housing to face towards the flange, the connector accessible through a hole in the flange.

19. An apparatus comprising:
an elongate housing having a length extending between a first end and a second end;
a first button to trigger X-ray equipment to prepare for an exposure;
a second button to trigger the X-ray equipment to implement the exposure, the first and second buttons spaced apart along the length of the elongate housing;
a first connector at the first end of the housing; and
a second connector at the second end of the housing, either of the first or second connectors to electrical couple with a third connector on a cable that is to be electrically coupled to the X-ray equipment.

20. An apparatus comprising:
an X-ray exposure switch assembly including:
a switch to trigger operation of an X-ray machine,
a connector electrical coupled to the switch, the connector to enable a cable to connect the X-ray exposure switch assembly to the X-ray machine, and
a housing to carry the switch and the connector; and
a cradle to support the X-ray exposure switch assembly on a floor to facilitate an operator to press the switch with a foot, the X-ray exposure switch assembly manually removable from the cradle by the operator without use of tools, the switch capable of being pressed by the operator when the X-ray exposure switch assembly is separated from the cradle.
